(19) United States
(12) Patent Application Publication
(10) Pub. No.: US 2012/0004294 A1
(43) Pub. Date: Jan. 5, 2012

(54) ANTITUMOR COMBINATION COMBINING AVE8062 AND DOCETAXEL

(75) Inventor: Michèle BESENVAL, Paris (FR)

(73) Assignee: SANOFI, Paris (FR)

(21) Appl. No.: 13/153,975

(22) Filed: Jun. 6, 2011

Related U.S. Application Data
(63) Continuation of application No. PCT/FR2009/052475, filed on Dec. 10, 2009.

(30) Foreign Application Priority Data
Dec. 12, 2008 (FR) 0806979

(51) Int. Cl. A61K 31/337; A61P 35/00

(52) U.S. Cl. 514/449

(57) ABSTRACT

The invention relates to a sequential antitumor combination of AVE8062, or a salt thereof, and docetaxel, characterized in that AVE8062 is administered to a patient in a dose of 10 to 50 mg/m2, and then, on a different day of the week, preferably after a 24-hour interval, docetaxel is administered in a dose of 50 to 120 mg/m2.

### CROSS-REFERENCE TO RELATED APPLICATIONS

This application is a continuation of International application No. PCT/FR2009/052475, filed Dec. 10, 2009, which claims the benefit of priority of French Patent Application No. 0806979, filed Dec. 12, 2008, both of which are incorporated herein by reference.

The present invention relates to an antitumor combination combining AVE8062 or a salt of AVE8062 and docetaxel in the treatment of solid tumors.

### BACKGROUND

Clinical Cancer Research 2004, 10, 415-427 compares vascular targeting agents (or VTAs) in the treatment of solid tumors. Among these, AVE8062A (AVE8062 hydrochloride) is administered alone at a weekly dose of 4.3-30 mg/m².

Proc. Am. Soc. Clin. Oncol. 2003, 22, 834, abstract 834 describes the administration of AVE8062A alone at weekly doses of 4.5, 6.0, 8.0, 11.5, 15.5, 22 and 30 mg/m². When AVE8062A is administered at too high a dose, cardiotoxicity was observed.

J. Clin. One. 2006, ASCO meeting, 2006:13074, Vol. 24, No. 18S describes, in abstract form, the combination of AVE8062A with oxaliplatin. This combination is also described in Jpn. J. Cancer Res. 1999, 90, 1016-1025.

WO 02/056692 describes combinations of a combretastatin A-4 and of two anticancer agents. Among the examples given, combretastatin A-4 at a dose of 1-100 mg/m² is combined with paclitaxel at a dose of 40-250 mg/m². WO 2006/078422 also describes a combination of a combretastatin at a dose of 1-100 mg/m² and of paclitaxel at a dose of 40-250 mg/m².

WO 02/074229 describes the combination of AVE8062 and of an anticancer agent chosen from taxanes, especially taxol or docetaxel, vinca alkaloids, alkylating agents and antimetabolites. The combination may consist in administering the two compounds at the same time or sequentially. The order of administration is not specified. The compounds may be administered orally, intravenously, subcutaneously or intramuscularly.

In the case of a taxane, this is administered by intraperitoneal injection at a dose between 1 and 10 mg/kg or intraveneous injection at a dose between 1 and 3 mg/kg. Examples are given of a combination, in mice, of AVE8062 at a dose of 150 mg/kg and of docetaxel at a dose of 109.6 mg/kg (AVE8062A/docetaxel ratio: 1.37). By taking a mouse [symbol] man conversion factor of 3 in the case of mice (see Freireich, E J “Quantitative comparison of toxicity of anticancer agents in mouse, rat, dog, monkey and man”, Cancer Chemother Rep. 1966, 50(4), 219-244), this results in a dose, in man, of 450 mg/m² of AVE8062 and 330 mg/m² of docetaxel.

Cancer Res. 2007, 67(19), 9337-9345 describes the combination of AVE8062A and of docetaxel in the treatment, in mice, of tumor cells of SKOV3ip1, HeyA8 or HeyA8-MDR type (ovarian cancer cells). The AVE8062A was administered, in mice, at a dose of 10, 30, 50 and 100 mg/kg (30-300 mg/m²) and the docetaxel at a dose of 2 or 1.4 mg/kg (6 or 4.2 mg/m²). The dose of 30 mg/kg is that recommended for AVE8062.

On the site www.clinicaltrials.gov, a phase I study (code NCT00719524) of an AVE8062+cisplatin (D1)/docetaxel (D2) combination in the treatment of patients having an advanced solid tumor is presented. No dose is specified.

Proc. Amer. Assoc. Cancer Res. 2005, Vol. 46, abstract#3425 (In vivo synergy between docetaxel and AVE8062A, a tumor vasculature targeting agent) describes the combination of AVE8062A and of docetaxel administered to mice bearing an MA13/C mammary tumor. The highest non-toxic dose (HNTD) which was found for this combination is 37.5 mg/kg/injection of AVE8062A and 54.8 mg/kg/injection of docetaxel (i.e. a docetaxel/AVE8062A ratio of 1.461). The present invention describes a combination intended to be administered to human patients.

### BRIEF DESCRIPTION OF THE INVENTION

The invention relates to an antitumor and sequential combination of AVE8062 or of an AVE8062 salt and of docetaxel, characterized in that the AVE8062 is administered to a patient at a dose between 10 and 50 mg/m², then on a different day of the week, preferably after an interval of 24 hours, the docetaxel is administered at a dose between 50 and 120 mg/m².

The dose of AVE8062 or of the AVE8062 salt is rather 20-40 mg/m², rather 30-40 mg/m². The dose of docetaxel is rather 50-100 mg/m², rather 60-80 mg/m². The dose of AVE8062 or of the AVE8062 salt may be 35 mg/m² and that of docetaxel 75 mg/m².

The AVE8062 or the AVE8062 salt and the docetaxel may be administered by perfusion.

The invention also relates to a combination intended to be administered to a patient during a cycle comprising an administration of AVE8062 or of an AVE8062 salt that marks the start of the cycle, then an administration of docetaxel, characterized in that the AVE8062 or the AVE8062 salt is administered first, then on a different day of the week, preferably after an interval of 24 hours, the docetaxel is administered, the doses of AVE8062 and of docetaxel being as defined in one of claims 1 to 4. The cycle may be repeated, the interval between two administrations of AVE8062 or of the AVE8062 salt ranges from 1 to 4 weeks, preferably 3 weeks.

The invention also relates to the use of AVE8062 or of an AVE8062 salt and of docetaxel for the preparation of an antitumor combination as defined in one of claims 1 to 10. The invention also relates to the use of AVE8062 or of an AVE8062 salt for the preparation of an antitumor combination as defined in one of claims 1 to 10.

The combination makes it possible to treat a solid tumor. It makes it possible to treat breast cancer, ovarian cancer, esophageal cancer, pancreatic cancer, cancer of the muscle tissue or of the soft tissue, head/neck cancer, bladder cancer, liver cancer, prostate cancer, ovarian cancer or skin cancer.

### DETAILED DESCRIPTION OF THE INVENTION

Regarding AVE8062, this has the formula:

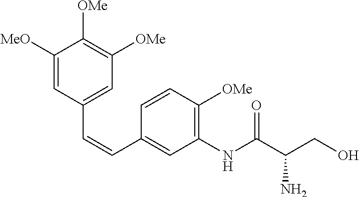

and has the chemical name (Z)—N-[2-methoxy-5-[2-(3,4,5 trimethoxyphenyl)vinyl]phenyl]-L-serinamide. AVE8062A denotes the hydrochloride of AVE8062.

AVE8062 may be prepared according to the process described in WO 03/084919. In the context of the protocol used, AVE8062A was used; this compound is packaged in the form of a vial containing an aqueous solution of the active principle. An amount of 25 mg of AVE8062A approximately is withdrawn from the vial, then diluted in a perfusion bag before being administered to the patient. The concentration of AVE8062A in the bag is between 0.012 mg/ml and 1.62 mg/ml. The perfusion volume administered to each patient depends on the patient.

Regarding docetaxel, this is sold under the trademark Taxotere® by Sanofi-Aventis. It has the chemical formula:

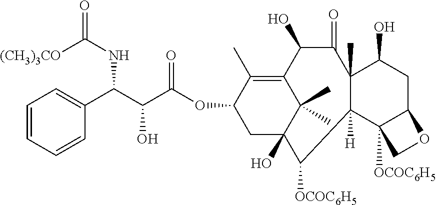

It may be in a form having the CAS No. 114977-28-5 or 148408-66-6 (trihydrate). The preparation of docetaxel is described, for example, in EP 0253738, EP 0253739 and WO 92/09589.

In the context of the protocol used, the docetaxel was packaged in the form of a vial containing anhydrous docetaxel in polysorbate 80 at a concentration of 40 mg/ml. It is possible to use a vial containing 20 mg of docetaxel (0.5 ml) that is then diluted with the contents of a vial (1.98 ml) of an aqueous solution of ethanol at 13% w/w so as to obtain a premix solution having a final docetaxel concentration of 10 mg/ml. It is also possible to use a vial containing 80 mg of docetaxel (2 ml) that is then diluted with the contents of a vial (7.33 ml) of an aqueous solution of ethanol at 13% w/w so as to obtain a premix solution having a final docetaxel concentration of 10 mg/ml.

The premix solution is itself then rediluted in a perfusion bag containing glucose or sodium chloride. The perfusion volume administered to each patient depends on the patient.

Regarding the antitumor combination, this consists in sequentially administering, preferably by perfusion, the AVE8062 or an AVE8062 salt, at a dose between 10 and 50 mg/m², then on a different day of the week, preferably after an interval of 24 hours, the docetaxel at a dose between 50 and 120 mg/m². It is preferable to sequentially combine the two compounds in this order, namely first the AVE8062 or the AVE8062 salt, then the docetaxel.

Preferably, the dose of AVE8062 or of the AVE8062 salt is 20-40 mg/m², rather 30-40 mg/m² and/or the dose of docetaxel is 50-100 mg/m², rather 60-80 mg/m². One combination may be, for example, 35 mg/m² of AVE8062 or of the AVE8062 salt and 75 mg/m² of docetaxel.

### Results

The protocol consisted in administering a combination of AVE8062A and of docetaxel to patients having an advanced solid tumor. The AVE8062A is administered by perfusion over a period of 30 min approximately and the next day, the docetaxel is administered by perfusion over a period of one hour approximately. This AVE8062A/docetaxel cycle is then repeated every three weeks.

Patients: median age: 53 years old (range 28-71 years old); 39 patients, 14 men/25 women; main tumor: breast (12 patients) and esophagus (8 patients).

TABLE I

| | |
|---|---|
| Number of patients treated | 39 |

| Sex | Male | 14 | (35.9%) |
|---|---|---|---|
| | Female | 25 | (64.1%) |
| median age (years) [range] | | 53 | [28-71] |
| ECOG PS | 0 | 15 | (38.5%) |
| | 1 | 24 | (61.5%) |
| Type of tumor | breast | 12 | (30.8%) |
| | esophagus | 8 | (20.5%) |
| | muscle/soft tissue | 5 | (12.8%) |
| | pancreas | 5 | (12.8%) |
| | other^{a} | 9 | (23.1%) |
| Prior chemotherapy | Prior chemotherapy (CT) | 36 | (92.3%) |
| | Median number of CT | 1 | [0-7] |
| | regimens for an advanced | | |
| | tumor [range] | | |
| | Prior treatment with taxanes | 18 | (46.1%) |
| | for an advanced tumor | 3 | (7.7%) |
| | and a refractory tumor^{b} | | |

^{a}includes head/neck cancer or cancer of unknown origin (2 patients each), and bladder, liver, prostate, ovarian and skin cancers (1 patient each).
^{b}tumor in progression during taxane treatment

TABLE II

| | | | | Number of | Number of |
|---|---|---|---|---|---|
| | | | | patients | patients |
| | | | | with DLT | with DLT at |
| | AVE8062A | docetaxel | | at the | a subsequent |
| dose¹ | [mg/m²] | [mg/m²] | Nₚₐₜᵢₑₙₜₛ | 1^{st} cycle | cycle |
| I | 11.5 | 75 | 3 | none | none |
| II | 15.5 | 75 | 5 | none | 1 gr 4 > 5 days |
| | | | | | (C2) neutropenia |
| III | 20 | 75 | 3 | none | neutropenic |
| | | | | | sepsis with gr 3 |
| | | | | | respiratory |
| | | | | | failure (C4) |
| IV | 25 | 75 | 6 | 1 neutro- | 1 gr 3 (C10, 11, |
| | | | | penic | 12) nail toxicity |
| | | | | infection | 1 gr 3 (C6) |
| | | | | | fistula |
| V | 30 | 75 | 3 | none | |
| V | 35 | 75 | 13 | none | 1 gr 3 (C2) |
| | | | | | ALAT |
| VI | 42 | 75 | 6 | grade 3 | 1 gr 3 (C2) |
| | | | | headaches | fatigue |
| | | | | 1 grade 3 | |
| | | | | fatigue | |

¹median cycle number: 3 (1-14)

These combinations did not lead to any severe cardiotoxic effect.

TABLE III

| dose | | | | | | | | |
|---|---|---|---|---|---|---|---|---|
| AVE8062A | 11.5 | 15.5 | 20 | 25 | 30 | 35 | 42 | total |
| [mg/m²] | N = 3 | N = 5 | N = 3 | N = 6 | N = 3 | N = 13 | N = 6 | N = 39 |
| Evaluable | 3 | 5 | 3 | 6 | 2 | 11 | 6 | 36 |
| number (N) | | | | | | | | (92.3%) |
| Partial | 0 | 1^{a} | 0 | 2^{a} | 0 | 0 | 0 | 3 |
| response | | | | | | | | (7.7%) |
| Stable | 1 | 1 | 3 | 2 | 2 | 7^{b} | 2 | 18 |
| disease | | | | | | | | (50%) |
| Tumor | 2 | 3 | 0 | 2 | 0 | 4 | 4 | 15 |
| progression | | | | | | | | (46.2%) |

Regarding the tumor, this may be a solid tumor, especially a solid tumor in an adult or in a child. The combination makes it possible to treat breast cancer, ovarian cancer, esophageal cancer, pancreatic cancer, cancer of the muscle tissue or of the soft tissue, head/neck cancer, bladder cancer, liver cancer, prostate cancer, ovarian cancer or skin cancer.

What is claimed is:

1. An antitumor and sequential combination of AVE8062 or of an AVE8062 salt and of docetaxel, characterized in that the AVE8062 is administered to a patient at a dose between 10 and 50 mg/m², then on a different day of the week, preferably after an interval of 24 hours, the docetaxel is administered at a dose between 50 and 120 mg/m².

2. The combination as claimed in claim 1, in which the dose of AVE8062 or of the AVE8062 salt is 20-40 mg/m² or 30-40 mg/m².

3. The combination as claimed in claim 1, in which the dose of docetaxel is 50-100 mg/m² or 60-80 mg/m².

4. The combination as claimed in claim 2, in which the dose of docetaxel is 50-100 mg/m² or 60-80 mg/m².

5. The combination as claimed in claim 1, in which the dose of AVE8062 or of the AVE8062 salt is 35 mg/m² and that of docetaxel is 75 mg/m².

6. The combination as claimed in claim 1, in which the AVE8062 or the AVE8062 salt and the docetaxel are administered by perfusion.

7. A combination intended to be administered to a patient during a cycle comprising an administration of AVE8062 or of an AVE8062 salt that marks the start of the cycle, then an administration of docetaxel, characterized in that the AVE8062 or the AVE8062 salt is administered first, then on a different day of the week, preferably after an interval of 24 hours, the docetaxel is administered, the doses of AVE8062 and of docetaxel being as defined in claim 1.

8. The combination as claimed in claim 7, characterized in that the cycle is repeated, the interval between two administrations of AVE8062 or of the AVE8062 salt ranges from 1 to 4 weeks, preferably 3 weeks.

9. A method of treating a patient with cancer, the method comprising administering to the patient the combination as claimed in claim 1.

10. The method as claimed in claim 9, wherein the cancer treated is a solid tumor.

11. The method as claimed in claim 10, wherein the solid tumor is not a solid breast tumor.

12. The method as claimed in claim 9, wherein the cancer is selected from the group consisting of breast cancer, ovarian cancer, esophageal cancer, pancreatic cancer, cancer of the muscle tissue or of the soft tissue, head/neck cancer, bladder cancer, liver cancer, prostate cancer, ovarian cancer and skin cancer.

13. The method as claimed in claim 9, wherein the dose of AVE8062 or of the AVE8062 salt is 20-40 mg/m² or 30-40 mg/m².

14. The method as claimed in claim 9, wherein the dose of docetaxel is 50-100 mg/m² or 60-80 mg/m².

15. The method as claimed in claim 13, wherein the dose of docetaxel is 50-100 mg/m² or 60-80 mg/m².

16. The method as claimed in claim 9, wherein the dose of AVE8062 or of the AVE8062 salt is 35 mg/m² and that of docetaxel is 75 mg/m².

17. The method as claimed in claim 9, wherein the AVE8062 or the AVE8062 salt and the docetaxel are administered by perfusion.

18. The method as claimed in claim 9, wherein administration of AVE8062 or of an AVE8062 salt marks the start of a cycle.

19. The method as claimed in claim 18, wherein the cycle is repeated and the interval between two administrations of AVE8062 or of the AVE8062 salt ranges from 1 to 4 weeks, preferably 3 weeks.
